SEQUENCE LISTING (86 sequences)

SEQ ID NO: 1 - DNA, 1092 bp, synthetic construct
  source 1..1092: mol_type = other DNA
gcggtgattaccccgaaccgccgcgtgcgcaaaaccccgtttaccccgggcgtggaagcggaaggcgtgaaaggctataccgtgtataaccacatgctgctgccgaccgtgtttgaaagtgtggaagccgatgcgagccatctgaaaaaacatgtgcaagtgtgggatgtgagctgcgaacgccaagtgctgattaaaggcccggatgcgctgcgcctgatgaaaatgctgagcccgcgcgatatggataaaatgcaagcggatcagtgctattatgtgccgattgtggatcagaacggcggcatgctgaacgatccggtggcgattaaactggcggatgatcattattggctgagcgtggcggatggcgatctgtggcagtttgcgctgggcattgtgctggcgctggaactggatgtggaagtgagcgaaccggatgtgagcccgctggcggtgcaaggcccgcgcgcggatgatctgatggcgcgcgtgtttggcgaagatgtgcgcgatattaaattttttcgctataaacgcctgccgtttgaagatacgagctttgtgattgcgcgcagcggctggagcaaacaaggcggctttgaaatttatgtggaaggcgcggattatggcatgccgctgtgggataccctgtttgcgaacggccgcgatctgaacgtgcgcgcgggctgcccgaacggcattgaacgcattgaaagcggcctgctgagctttggcagcgatatgcgccgcgaaaacaccccgtatgaatgcggcctggaacgcttttgcaacagcccgctggattatattggcaaagcggcgctggcggatcaagcggaaaacggcccgccgcgtcagattcgcccgctgaccattgaaggcgaaattggcccgtgcaccgaaagctggccgctgatggccgaaggtcgccaagtgggccaagtggcgagcgcggcgtatagcccggaatttgaagtgaacgtggcgattggcatggtggatcgcagccattgggcgccgggcacccgcattgaactgatgaccgaacatggcatgcgctgggcgattgtgcaagaaaaattttggcgctaa

SEQ ID NO: 2 - protein, 364 aa, synthetic construct
MAVITPNRRVRKTPFTPGVEAEGVKGYTVYNHMLLPTVFESVEADASHLKKHVQVWDVSCERQVLIKGPDALRLMKMLSPRDMDKMQADQCYYVPIVDQNGGMLNDPVAIKLADDHYWLSVADGDLWQFALGIVLALELDVEVSEPDVSPLAVQGPRADDLMARVFGEDVRDIKFFRYKRLPFEDTSFVIARSGWSKQGGFEIYVEGADYGMPLWDTLFANGRDLNVRAGCPNGIERIESGLLSFGSDMRRENTPYECGLERFCNSPLDYIGKAALADQAENGPPRQIRPLTIEGEIGPCTESWPLMAEGRQVGQVASAAYSPEFEVNVAIGMVDRSHWAPGTRIELMTEHGMRWAIVQEKFWR

SEQ ID NO: 3 - protein, 364 aa, synthetic construct
MAVITPNRRVRKTPFTPGVEAEGVKGYTVYNHMLLPTVFESVEADASHLKKHVQVWDVSCERQVLIKGPDALRLMKMLSPRDMDKMQADQCYYVPIVDQNGGMLNDPVAIKLADDHYWLSVADGDLWQFALGIVLALELDVEVSEPDVSPLAVQGPRADDLMARVFGEDVRDIKFFRYKRLPFEDTSFVIARSGWSKQGGFEIYVEGADYGMPLWDTLFANGRDLNVRAGCPNGIERIESGLLSFGSDMRRENTPYECGLERYCNSPLDYIGKAALADQAENGPPRQIRPLTIEGEIGPCTESWPLMAEGRQVGQVASAAYSPEFEVNVAIGMVDRSHWAPGTRIELMTEHGMRWAIVQEKFWR

SEQ ID NO: 4 - protein, 364 aa, synthetic construct
MAVITPNRRVRKTPFTPGVEAEGVKGYTVYNHMLLPTVFESVEADASHLKKHVQVWDVSCERQVLIKGPDALRLMKMLSPRDMDKMQADQCYYVPIVDQNGGMLNDPVAIKLADDHYWLSIADGDLWQFALGIVLALELDVEVSEPDVSPLAVQGPRADDLMARVFGEDVRDIKFFRYKRLPFEDTSFVIARSGWSKQGGFEIYVEGADYGMPLWDTLFANGRDLNVRAGCPNGIERIESGLLSFGSDMRRENTPYECGLERFCNSPLDYIGKAALADQAENGPPRQIRPLTIEGEIGPCTESWPLMAEGRQVGQVASAAYSPEFEVNVAIGMVDRSHWAPGTRIELMTEHGMRWAIVQEKFWR

SEQ ID NO: 5 - protein, 364 aa, synthetic construct
MAVITPNRRVRKTPFTPGVEAEGVKGYTVYNHMLLPTVFESVEADASHLKKHVQVWDVSCERQVLIKGPDALRLMKMLSPRDMDKMQADQCYYVPIVDQNGGMLNDPVAIKLADDHYWLSVSDGDLWQFALGIVLALELDVEVSEPDVSPLAVQGPRADDLMARVFGEDVRDIKFFRYKRLPFEDTSFVIARSGWSKQGGFEIYVEGADYGMPLWDTLFANGRDLNVRAGCPNGIERIESGLLSFGSDMRRENTPYECGLERFCNSPLDYIGKAALADQAENGPPRQIRPLTIEGEIGPCTESWPLMAEGRQVGQVASAAYSPEFEVNVAIGMVDRSHWAPGTRIELMTEHGMRWAIVQEKFWR

SEQ ID NO: 6 - protein, 364 aa, synthetic construct
MAVITPNRRVRKTPFTPGVEAEGVKGYTVYNHMLLPTVFESVEADASHLKKHVQVWDVSCERQVLIKGPDALRLMKMLSPRDMDKMQADQCYYVPIVDQNGGMLNDPVAIKLADDHYWLSSADGDLWQFALGIVLALELDVEVSEPDVSPLAVQGPRADDLMARVFGEDVRDIKFFRYKRLPFEDTSFVIARSGWSKQGGFEIYVEGADYGMPLWDTLFANGRDLNVRAGCPNGIERIESGLLSFGSDMRRENTPYECGLERFCNSPLDYIGKAALADQAENGPPRQIRPLTIEGEIGPCTESWPLMAEGRQVGQVASAAYSPEFEVNVAIGMVDRSHWAPGTRIELMTEHGMRWAIVQEKFWR

SEQ ID NO: 7 - protein, 364 aa, synthetic construct
MAVITPNRRVRKTPFTPGVEAEGVKGYTVYNHMLLPTVFESVEADASHLKKHVQVWDVSCERQVLIKGPDALRLMKMLSPRDMDKMQADQCYYVPIVDQNGGMLNDIVAIKLADDHYWLSVADGDLWQFALGIVLALELDVEVSEPDVSPLAVQGPRADDLMARVFGEDVRDIKFFRYKRLPFEDTSFVIARSGWSKQGGFEIYVEGADYGMPLWDTLFANGRDLNVRAGCPNGIERIESGLLSFGSDMRRENTPYECGLERFCNSPLDYIGKAALADQAENGPPRQIRPLTIEGEIGPCTESWPLMAEGRQVGQVASAAYSPEFEVNVAIGMVDRSHWAPGTRIELMTEHGMRWAIVQEKFWR

SEQ ID NO: 8 - protein, 364 aa, synthetic construct
MAVITPNRRVRKTPFTPGVEAEGVKGYTVYNHMLLPTVFESVEADASHLKKHVQVWDVSCERQVLIKGPDALRLMKMLSPRDMDKMQADQCYYVPIVDQNGGMLNDPVAIKLADDHYWLSVADGDLWQFALGIVLALELDVEVSEPDVSPLAVQGPRADDLMARVFGEDVRDIKFFRYKRLPFEDTSFVIARSGWSKQGGFEIYVEGADYGMPLWDTLFANGRDLNVRAGCPNGIERIESGLLSFGSDMRRENTPYECGLERICNSPLDYIGKAALADQAENGPPRQIRPLTIEGEIGPCTESWPLMAEGRQVGQVASAAYSPEFEVNVAIGMVDRSHWAPGTRIELMTEHGMRWAIVQEKFWR

SEQ ID NO: 9 - protein, 364 aa, synthetic construct
MAVITPNRRVRKTPFTPGVEAEGVKGYTVYNHMLLPTVFESVEADASHLKKHVQVWDVSCERQVLIKGPDALRLMKMLSPRDMDKMQADQCYYVPIVDQNGGMLNDPVAIKLADDHYWLSVADGDLWQFALGIVLALELDVEVSEPDVSPLAVQGPRADDLMARVFGEDVRDIKFFRYKRLPFEDTSFVIARSGWSKQGGFEIYVEGADYGMPLWDTLFANGRDLNVRAGCPNGIERIESGLLSYGSDMRRENTPYECGLERFCNSPLDYIGKAALADQAENGPPRQIRPLTIEGEIGPCTESWPLMAEGRQVGQVASAAYSPEFEVNVAIGMVDRSHWAPGTRIELMTEHGMRWAIVQEKFWR

SEQ ID NO: 10 - DNA, 1092 bp, synthetic construct
  source 1..1092: mol_type = other DNA
gcggtgattaccccgaaccgccgcgtgcgcaaaaccccgtttaccccgggcgtggaagcggaaggcgtgaaaggctataccgtgtataaccacatgctgctgccgaccgtgtttgaaagtgtggaagccgatgcgagccatctgaaaaaacatgtgcaagtgtgggatgtgagctgcgaacgccaagtgctgattaaaggcccggatgcgctgcgcctgatgaaaatgctgagcccgcgcgatatggataaaatgcaagcggatcagtgctattatgtgccgattgtggatcagaacggcggcatgctgaacgatccggtggcgattaaactggcggatgatcattattggctgagcgtggcggatggcgatctgtggcagtttgcgctgggcattgtgctggcgctggaactggatgtggaagtgagcgaaccggatgtgagcccgctggcggtgcaaggcccgcgcgcggatgatctgatggcgcgcgtgtttggcgaagatgtgcgcgatattaaattttttcgctataaacgcctgccgtttgaagatacgagctttgtgattgcgcgcagcggctggagcaaacaaggcggctttgaaatttatgtggaaggcgcggattatggcatgccgctgtgggataccctgtttgcgaacggccgcgatctgaacgtgcgcgcgggctgcccgaacggcattgaacgcattgaaagcggcctgctgagctttggcagcgatatgcgccgcgaaaacaccccgtatgaatgcggcctggaacgctattgcaacagcccgctggattatattggcaaagcggcgctggcggatcaagcggaaaacggcccgccgcgtcagattcgcccgctgaccattgaaggcgaaattggcccgtgcaccgaaagctggccgctgatggccgaaggtcgccaagtgggccaagtggcgagcgcggcgtatagcccggaatttgaagtgaacgtggcgattggcatggtggatcgcagccattgggcgccgggcacccgcattgaactgatgaccgaacatggcatgcgctgggcgattgtgcaagaaaaattttggcgctaa

SEQ ID NO: 11 - DNA, 1092 bp, synthetic construct
  source 1..1092: mol_type = other DNA
gcggtgattaccccgaaccgccgcgtgcgcaaaaccccgtttaccccgggcgtggaagcggaaggcgtgaaaggctataccgtgtataaccacatgctgctgccgaccgtgtttgaaagtgtggaagccgatgcgagccatctgaaaaaacatgtgcaagtgtgggatgtgagctgcgaacgccaagtgctgattaaaggcccggatgcgctgcgcctgatgaaaatgctgagcccgcgcgatatggataaaatgcaagcggatcagtgctattatgtgccgattgtggatcagaacggcggcatgctgaacgatccggtggcgattaaactggcggatgatcattattggctgagcattgcggatggcgatctgtggcagtttgcgctgggcattgtgctggcgctggaactggatgtggaagtgagcgaaccggatgtgagcccgctggcggtgcaaggcccgcgcgcggatgatctgatggcgcgcgtgtttggcgaagatgtgcgcgatattaaattttttcgctataaacgcctgccgtttgaagatacgagctttgtgattgcgcgcagcggctggagcaaacaaggcggctttgaaatttatgtggaaggcgcggattatggcatgccgctgtgggataccctgtttgcgaacggccgcgatctgaacgtgcgcgcgggctgcccgaacggcattgaacgcattgaaagcggcctgctgagctttggcagcgatatgcgccgcgaaaacaccccgtatgaatgcggcctggaacgcttttgcaacagcccgctggattatattggcaaagcggcgctggcggatcaagcggaaaacggcccgccgcgtcagattcgcccgctgaccattgaaggcgaaattggcccgtgcaccgaaagctggccgctgatggccgaaggtcgccaagtgggccaagtggcgagcgcggcgtatagcccggaatttgaagtgaacgtggcgattggcatggtggatcgcagccattgggcgccgggcacccgcattgaactgatgaccgaacatggcatgcgctgggcgattgtgcaagaaaaattttggcgctaa

SEQ ID NO: 12 - DNA, 1092 bp, synthetic construct
  source 1..1092: mol_type = other DNA
gcggtgattaccccgaaccgccgcgtgcgcaaaaccccgtttaccccgggcgtggaagcggaaggcgtgaaaggctataccgtgtataaccacatgctgctgccgaccgtgtttgaaagtgtggaagccgatgcgagccatctgaaaaaacatgtgcaagtgtgggatgtgagctgcgaacgccaagtgctgattaaaggcccggatgcgctgcgcctgatgaaaatgctgagcccgcgcgatatggataaaatgcaagcggatcagtgctattatgtgccgattgtggatcagaacggcggcatgctgaacgatccggtggcgattaaactggcggatgatcattattggctgagcgtgagcgatggcgatctgtggcagtttgcgctgggcattgtgctggcgctggaactggatgtggaagtgagcgaaccggatgtgagcccgctggcggtgcaaggcccgcgcgcggatgatctgatggcgcgcgtgtttggcgaagatgtgcgcgatattaaattttttcgctataaacgcctgccgtttgaagatacgagctttgtgattgcgcgcagcggctggagcaaacaaggcggctttgaaatttatgtggaaggcgcggattatggcatgccgctgtgggataccctgtttgcgaacggccgcgatctgaacgtgcgcgcgggctgcccgaacggcattgaacgcattgaaagcggcctgctgagctttggcagcgatatgcgccgcgaaaacaccccgtatgaatgcggcctggaacgcttttgcaacagcccgctggattatattggcaaagcggcgctggcggatcaagcggaaaacggcccgccgcgtcagattcgcccgctgaccattgaaggcgaaattggcccgtgcaccgaaagctggccgctgatggccgaaggtcgccaagtgggccaagtggcgagcgcggcgtatagcccggaatttgaagtgaacgtggcgattggcatggtggatcgcagccattgggcgccgggcacccgcattgaactgatgaccgaacatggcatgcgctgggcgattgtgcaagaaaaattttggcgctaa

SEQ ID NO: 13 - DNA, 1092 bp, synthetic construct
  source 1..1092: mol_type = other DNA
gcggtgattaccccgaaccgccgcgtgcgcaaaaccccgtttaccccgggcgtggaagcggaaggcgtgaaaggctataccgtgtataaccacatgctgctgccgaccgtgtttgaaagtgtggaagccgatgcgagccatctgaaaaaacatgtgcaagtgtgggatgtgagctgcgaacgccaagtgctgattaaaggcccggatgcgctgcgcctgatgaaaatgctgagcccgcgcgatatggataaaatgcaagcggatcagtgctattatgtgccgattgtggatcagaacggcggcatgctgaacgatccggtggcgattaaactggcggatgatcattattggctgagcagcgcggatggcgatctgtggcagtttgcgctgggcattgtgctggcgctggaactggatgtggaagtgagcgaaccggatgtgagcccgctggcggtgcaaggcccgcgcgcggatgatctgatggcgcgcgtgtttggcgaagatgtgcgcgatattaaattttttcgctataaacgcctgccgtttgaagatacgagctttgtgattgcgcgcagcggctggagcaaacaaggcggctttgaaatttatgtggaaggcgcggattatggcatgccgctgtgggataccctgtttgcgaacggccgcgatctgaacgtgcgcgcgggctgcccgaacggcattgaacgcattgaaagcggcctgctgagctttggcagcgatatgcgccgcgaaaacaccccgtatgaatgcggcctggaacgcttttgcaacagcccgctggattatattggcaaagcggcgctggcggatcaagcggaaaacggcccgccgcgtcagattcgcccgctgaccattgaaggcgaaattggcccgtgcaccgaaagctggccgctgatggccgaaggtcgccaagtgggccaagtggcgagcgcggcgtatagcccggaatttgaagtgaacgtggcgattggcatggtggatcgcagccattgggcgccgggcacccgcattgaactgatgaccgaacatggcatgcgctgggcgattgtgcaagaaaaattttggcgctaa

SEQ ID NO: 14 - DNA, 1092 bp, synthetic construct
  source 1..1092: mol_type = other DNA
gcggtgattaccccgaaccgccgcgtgcgcaaaaccccgtttaccccgggcgtggaagcggaaggcgtgaaaggctataccgtgtataaccacatgctgctgccgaccgtgtttgaaagtgtggaagccgatgcgagccatctgaaaaaacatgtgcaagtgtgggatgtgagctgcgaacgccaagtgctgattaaaggcccggatgcgctgcgcctgatgaaaatgctgagcccgcgcgatatggataaaatgcaagcggatcagtgctattatgtgccgattgtggatcagaacggcggcatgctgaacgatattgtggcgattaaactggcggatgatcattattggctgagcgtggcggatggcgatctgtggcagtttgcgctgggcattgtgctggcgctggaactggatgtggaagtgagcgaaccggatgtgagcccgctggcggtgcaaggcccgcgcgcggatgatctgatggcgcgcgtgtttggcgaagatgtgcgcgatattaaattttttcgctataaacgcctgccgtttgaagatacgagctttgtgattgcgcgcagcggctggagcaaacaaggcggctttgaaatttatgtggaaggcgcggattatggcatgccgctgtgggataccctgtttgcgaacggccgcgatctgaacgtgcgcgcgggctgcccgaacggcattgaacgcattgaaagcggcctgctgagctttggcagcgatatgcgccgcgaaaacaccccgtatgaatgcggcctggaacgcttttgcaacagcccgctggattatattggcaaagcggcgctggcggatcaagcggaaaacggcccgccgcgtcagattcgcccgctgaccattgaaggcgaaattggcccgtgcaccgaaagctggccgctgatggccgaaggtcgccaagtgggccaagtggcgagcgcggcgtatagcccggaatttgaagtgaacgtggcgattggcatggtggatcgcagccattgggcgccgggcacccgcattgaactgatgaccgaacatggcatgcgctgggcgattgtgcaagaaaaattttggcgctaa

SEQ ID NO: 15 - DNA, 1092 bp, synthetic construct
  source 1..1092: mol_type = other DNA
gcggtgattaccccgaaccgccgcgtgcgcaaaaccccgtttaccccgggcgtggaagcggaaggcgtgaaaggctataccgtgtataaccacatgctgctgccgaccgtgtttgaaagtgtggaagccgatgcgagccatctgaaaaaacatgtgcaagtgtgggatgtgagctgcgaacgccaagtgctgattaaaggcccggatgcgctgcgcctgatgaaaatgctgagcccgcgcgatatggataaaatgcaagcggatcagtgctattatgtgccgattgtggatcagaacggcggcatgctgaacgatccggtggcgattaaactggcggatgatcattattggctgagcgtggcggatggcgatctgtggcagtttgcgctgggcattgtgctggcgctggaactggatgtggaagtgagcgaaccggatgtgagcccgctggcggtgcaaggcccgcgcgcggatgatctgatggcgcgcgtgtttggcgaagatgtgcgcgatattaaattttttcgctataaacgcctgccgtttgaagatacgagctttgtgattgcgcgcagcggctggagcaaacaaggcggctttgaaatttatgtggaaggcgcggattatggcatgccgctgtgggataccctgtttgcgaacggccgcgatctgaacgtgcgcgcgggctgcccgaacggcattgaacgcattgaaagcggcctgctgagctttggcagcgatatgcgccgcgaaaacaccccgtatgaatgcggcctggaacgcatttgcaacagcccgctggattatattggcaaagcggcgctggcggatcaagcggaaaacggcccgccgcgtcagattcgcccgctgaccattgaaggcgaaattggcccgtgcaccgaaagctggccgctgatggccgaaggtcgccaagtgggccaagtggcgagcgcggcgtatagcccggaatttgaagtgaacgtggcgattggcatggtggatcgcagccattgggcgccgggcacccgcattgaactgatgaccgaacatggcatgcgctgggcgattgtgcaagaaaaattttggcgctaa

SEQ ID NO: 16 - DNA, 1092 bp, synthetic construct
  source 1..1092: mol_type = other DNA
gcggtgattaccccgaaccgccgcgtgcgcaaaaccccgtttaccccgggcgtggaagcggaaggcgtgaaaggctataccgtgtataaccacatgctgctgccgaccgtgtttgaaagtgtggaagccgatgcgagccatctgaaaaaacatgtgcaagtgtgggatgtgagctgcgaacgccaagtgctgattaaaggcccggatgcgctgcgcctgatgaaaatgctgagcccgcgcgatatggataaaatgcaagcggatcagtgctattatgtgccgattgtggatcagaacggcggcatgctgaacgatccggtggcgattaaactggcggatgatcattattggctgagcgtggcggatggcgatctgtggcagtttgcgctgggcattgtgctggcgctggaactggatgtggaagtgagcgaaccggatgtgagcccgctggcggtgcaaggcccgcgcgcggatgatctgatggcgcgcgtgtttggcgaagatgtgcgcgatattaaattttttcgctataaacgcctgccgtttgaagatacgagctttgtgattgcgcgcagcggctggagcaaacaaggcggctttgaaatttatgtggaaggcgcggattatggcatgccgctgtgggataccctgtttgcgaacggccgcgatctgaacgtgcgcgcgggctgcccgaacggcattgaacgcattgaaagcggcctgctgagctatggcagcgatatgcgccgcgaaaacaccccgtatgaatgcggcctggaacgcttttgcaacagcccgctggattatattggcaaagcggcgctggcggatcaagcggaaaacggcccgccgcgtcagattcgcccgctgaccattgaaggcgaaattggcccgtgcaccgaaagctggccgctgatggccgaaggtcgccaagtgggccaagtggcgagcgcggcgtatagcccggaatttgaagtgaacgtggcgattggcatggtggatcgcagccattgggcgccgggcacccgcattgaactgatgaccgaacatggcatgcgctgggcgattgtgcaagaaaaattttggcgctaa

SEQ ID NO: 17 - DNA, 35 bp, synthetic construct
  source 1..35: mol_type = other DNA
ggaaggcgtgaaaggctataccatttataaccaca

SEQ ID NO: 18 - DNA, 27 bp, synthetic construct
  source 1..27: mol_type = other DNA
atagcctttcacgccttccgcttccac

SEQ ID NO: 19 - DNA, 38 bp, synthetic construct
  source 1..38: mol_type = other DNA
atgtgcaagtgtgggatgtgagcgcggaacgccaagtg

SEQ ID NO: 20 - DNA, 28 bp, synthetic construct
  source 1..28: mol_type = other DNA
atcccacacttgcacatgttttttcaga

SEQ ID NO: 21 - DNA, 33 bp, synthetic construct
  source 1..33: mol_type = other DNA
agcggcctgctgagctttggcaacgatatgcgc

SEQ ID NO: 22 - DNA, 25 bp, synthetic construct
  source 1..25: mol_type = other DNA
gctcagcaggccgctttcaatgcgt

SEQ ID NO: 23 - DNA, 43 bp, synthetic construct
  source 1..43: mol_type = other DNA
ctggcggatgatcattattggctgagcattgcggatggcgatc

SEQ ID NO: 24 - DNA, 37 bp, synthetic construct
  source 1..37: mol_type = other DNA
aataatgatcatccgccagtttaatcgccaccggatc

SEQ ID NO: 25 - DNA, 39 bp, synthetic construct
  source 1..39: mol_type = other DNA
gtgagcgaaccggatgtgagcattctggcggtgcaaggc

SEQ ID NO: 26 - DNA, 31 bp, synthetic construct
  source 1..31: mol_type = other DNA
acatccggttcgctcacttccacatccagtt

SEQ ID NO: 27 - DNA, 37 bp, synthetic construct
  source 1..37: mol_type = other DNA
tgtgattgcgcgcagcggctggcaaaaacaaggcggc

SEQ ID NO: 28 - DNA, 29 bp, synthetic construct
  source 1..29: mol_type = other DNA
cgctgcgcgcaatcacaaagctcgtatct

SEQ ID NO: 29 - DNA, 35 bp, synthetic construct
  source 1..35: mol_type = other DNA
gcattgaaagcggcctgctgagctatggcagcgat

SEQ ID NO: 30 - DNA, 39 bp, synthetic construct
  source 1..39: mol_type = other DNA
cagcaggccgctttcaatgcgttcaatgccgttcgggca

SEQ ID NO: 31 - DNA, 40 bp, synthetic construct
  source 1..40: mol_type = other DNA
agcggcctgctgagctttggcagcgattttcgccgcgaaa

SEQ ID NO: 32 - DNA, 38 bp, synthetic construct
  source 1..38: mol_type = other DNA
tgccaaagctcagcaggccgctttcaatgcgttcaatg

SEQ ID NO: 33 - DNA, 39 bp, synthetic construct
  source 1..39: mol_type = other DNA
ccccgtatgaatgcggcctggaacgctattgcaacagcc

SEQ ID NO: 34 - DNA, 35 bp, synthetic construct
  source 1..35: mol_type = other DNA
ccaggccgcattcatacggggtgttttcgcggcgc

SEQ ID NO: 35 - DNA, 37 bp, synthetic construct
  source 1..37: mol_type = other DNA
tgatcattattggctgagcgtggcggatggcgatctg

SEQ ID NO: 36 - DNA, 37 bp, synthetic construct
  source 1..37: mol_type = other DNA
tgatcattattggctgagcaktgcggatggcgatctg

SEQ ID NO: 37 - DNA, 37 bp, synthetic construct
  source 1..37: mol_type = other DNA
tgatcattattggctgagctatgcggatggcgatctg

SEQ ID NO: 38 - DNA, 37 bp, synthetic construct
  source 1..37: mol_type = other DNA
tgatcattattggctgagcgtgaktgatggcgatctg

SEQ ID NO: 39 - DNA, 37 bp, synthetic construct
  source 1..37: mol_type = other DNA
tgatcattattggctgagcgtgtatgatggcgatctg

SEQ ID NO: 40 - DNA, 37 bp, synthetic construct
  source 1..37: mol_type = other DNA
tgatcattattggctgagcaktaktgatggcgatctg

SEQ ID NO: 41 - DNA, 37 bp, synthetic construct
  source 1..37: mol_type = other DNA
tgatcattattggctgagctattatgatggcgatctg

SEQ ID NO: 42 - DNA, 37 bp, synthetic construct
  source 1..37: mol_type = other DNA
tgatcattattggctgagcakttatgatggcgatctg

SEQ ID NO: 43 - DNA, 37 bp, synthetic construct
  source 1..37: mol_type = other DNA
tgatcattattggctgagctataktgatggcgatctg

SEQ ID NO: 44 - DNA, 31 bp, synthetic construct
  source 1..31: mol_type = other DNA
cgtttatagcgaaaaaatttaatatcgcgca

SEQ ID NO: 45 - DNA, 31 bp, synthetic construct
  source 1..31: mol_type = other DNA
cgtttatagcgaaatmatttaatatcgcgca

SEQ ID NO: 46 - DNA, 31 bp, synthetic construct
  source 1..31: mol_type = other DNA
cgtttatagcgaaaatatttaatatcgcgca

SEQ ID NO: 47 - DNA, 31 bp, synthetic construct
  source 1..31: mol_type = other DNA
cgtttatagcgtmaaaatttaatatcgcgca

SEQ ID NO: 48 - DNA, 31 bp, synthetic construct
  source 1..31: mol_type = other DNA
cgtttatagcgtmatmatttaatatcgcgca

SEQ ID NO: 49 - DNA, 31 bp, synthetic construct
  source 1..31: mol_type = other DNA
cgtttatagcgataatatttaatatcgcgca

SEQ ID NO: 50 - DNA, 31 bp, synthetic construct
  source 1..31: mol_type = other DNA
cgtttatagcgtmaatatttaatatcgcgca

SEQ ID NO: 51 - DNA, 31 bp, synthetic construct
  source 1..31: mol_type = other DNA
cgtttatagcgatatmatttaatatcgcgca

SEQ ID NO: 52 - DNA, 30 bp, synthetic construct
  source 1..30: mol_type = other DNA
cagccaataatgatcatccgccagtttaat

SEQ ID NO: 53 - DNA, 37 bp, synthetic construct
  source 1..37: mol_type = other DNA
gtgagcgaaccggatgtgagcccgctggcggtgcaag

SEQ ID NO: 54 - DNA, 37 bp, synthetic construct
  source 1..37: mol_type = other DNA
gtgagcgaaccggatgtgagcaktctggcggtgcaag

SEQ ID NO: 55 - DNA, 37 bp, synthetic construct
  source 1..37: mol_type = other DNA
gtgagcgaaccggatgtgagctatctggcggtgcaag

SEQ ID NO: 56 - DNA, 32 bp, synthetic construct
  source 1..32: mol_type = other DNA
cttgtttgctccagccgctgcgcgcaatcaca

SEQ ID NO: 57 - DNA, 32 bp, synthetic construct
  source 1..32: mol_type = other DNA
cttgtttgctccagccatagcgcgcaatcaca

SEQ ID NO: 58 - DNA, 32 bp, synthetic construct
  source 1..32: mol_type = other DNA
cttgtttgctccagccaatgcgcgcaatcaca

SEQ ID NO: 59 - DNA, 32 bp, synthetic construct
  source 1..32: mol_type = other DNA
cttgtttgctccaamtgctgcgcgcaatcaca

SEQ ID NO: 60 - DNA, 32 bp, synthetic construct
  source 1..32: mol_type = other DNA
cttgtttgctccaatagctgcgcgcaatcaca

SEQ ID NO: 61 - DNA, 32 bp, synthetic construct
  source 1..32: mol_type = other DNA
cttgtttgctccaamtatagcgcgcaatcaca

SEQ ID NO: 62 - DNA, 32 bp, synthetic construct
  source 1..32: mol_type = other DNA
cttgtttgctccaataaatgcgcgcaatcaca

SEQ ID NO: 63 - DNA, 32 bp, synthetic construct
  source 1..32: mol_type = other DNA
cttgtttgctccaataatagcgcgcaatcaca

SEQ ID NO: 64 - DNA, 32 bp, synthetic construct
  source 1..32: mol_type = other DNA
cttgtttgctccaamtaatgcgcgcaatcaca

SEQ ID NO: 65 - DNA, 31 bp, synthetic construct
  source 1..31: mol_type = other DNA
tccggttcgctcacttccacatccagttcca

SEQ ID NO: 66 - DNA, 40 bp, synthetic construct
  source 1..40: mol_type = other DNA
cattgaacgcattgaaagcggcctgctgagctttggcagc

SEQ ID NO: 67 - DNA, 40 bp, synthetic construct
  source 1..40: mol_type = other DNA
cattgaacgcattgaaagcggcaktctgagctttggcagc

SEQ ID NO: 68 - DNA, 40 bp, synthetic construct
  source 1..40: mol_type = other DNA
cattgaacgcattgaaagcggctatctgagctttggcagc

SEQ ID NO: 69 - DNA, 40 bp, synthetic construct
  source 1..40: mol_type = other DNA
cattgaacgcattgaaagcggcctgaktagctttggcagc

SEQ ID NO: 70 - DNA, 40 bp, synthetic construct
  source 1..40: mol_type = other DNA
cattgaacgcattgaaagcggcctgtatagctttggcagc

SEQ ID NO: 71 - DNA, 40 bp, synthetic construct
  source 1..40: mol_type = other DNA
cattgaacgcattgaaagcggcakttatagctttggcagc

SEQ ID NO: 72 - DNA, 40 bp, synthetic construct
  source 1..40: mol_type = other DNA
cattgaacgcattgaaagcggctataktagctttggcagc

SEQ ID NO: 73 - DNA, 40 bp, synthetic construct
  source 1..40: mol_type = other DNA
cattgaacgcattgaaagcggctattatagctttggcagc

SEQ ID NO: 74 - DNA, 40 bp, synthetic construct
  source 1..40: mol_type = other DNA
cattgaacgcattgaaagcggcaktaktagctttggcagc

SEQ ID NO: 75 - DNA, 29 bp, synthetic construct
  source 1..29: mol_type = other DNA
cgggctgttgcaaaagcgttccaggccgc

SEQ ID NO: 76 - DNA, 29 bp, synthetic construct
  source 1..29: mol_type = other DNA
cgggctgttgcaamtgcgttccaggccgc

SEQ ID NO: 77 - DNA, 29 bp, synthetic construct
  source 1..29: mol_type = other DNA
cgggctgttgcaatagcgttccaggccgc

SEQ ID NO: 78 - DNA, 30 bp, synthetic construct
  source 1..30: mol_type = other DNA
gctttcaatgcgttcaatgccgttcgggca

SEQ ID NO: 79 - DNA, 38 bp, synthetic construct
  source 1..38: mol_type = other DNA
ggaaggcgtgaaaggctataccgtgaktaaccacatgc

SEQ ID NO: 80 - DNA, 70 bp, synthetic construct
  source 1..70: mol_type = other DNA
gtttaatcgccaccggatcgttcagcatgccgccgttctgatccacaatcggcacataatagcactgatc

SEQ ID NO: 81 - DNA, 70 bp, synthetic construct
  source 1..70: mol_type = other DNA
gtttaatcgccacamtatcgttcagcatgccgccgttctgatccacaatcggcacamtatagcactgatc

SEQ ID NO: 82 - DNA, 70 bp, synthetic construct
  source 1..70: mol_type = other DNA
gtttaatcgccacataatcgttcagcatgccgccgttctgatccacaatcggcacataatagcactgatc

SEQ ID NO: 83 - DNA, 70 bp, synthetic construct
  source 1..70: mol_type = other DNA
gtttaatcgccacataatcgttcagcatgccgccgttctgatccacaatcggcacamtatagcactgatc

SEQ ID NO: 84 - DNA, 70 bp, synthetic construct
  source 1..70: mol_type = other DNA
gtttaatcgccaccggatcgttcagcatgccgccgttctgatccacaatcggcacamtatagcactgatc

SEQ ID NO: 85 - DNA, 70 bp, synthetic construct
  source 1..70: mol_type = other DNA
gtttaatcgccacamtatcgttcagcatgccgccgttctgatccacaatcggcacataatagcactgatc

SEQ ID NO: 86 - DNA, 30 bp, synthetic construct
  source 1..30: mol_type = other DNA
agcctttcacgccttccgcttccacgcccg

(19) United States
(12) Patent Application Publication
(10) Pub. No.: US 2026/0117270 A1
(43) Pub. Date: Apr. 30, 2026

(54) TETRAHYDROFOLATE METHYLTRANSFERASE MUTANT, ENCODING GENE AND APPLICATIONS THEREOF

(71) Applicants: ZHEJIANG SHENGDA BIO-PHARM CO., LTD., Taizhou (CN); ZHEJIANG UNIVERSITY OF TECHNOLOGY, Hangzhou (CN); ZHEJIANG ZHULONG BIOTECHNOLOGY CO., LTD., Huzhou (CN)

(72) Inventors: Xiaolong CHEN, Taizhou (CN); Linjiang ZHU, Taizhou (CN); Yuxin WANG, Taizhou (CN); Chao LIANG, Taizhou (CN); Lingjun JU, Taizhou (CN); Yongxian FAN, Taizhou (CN)

(73) Assignees: ZHEJIANG SHENGDA BIO-PHARM CO., LTD., Taizhou (CN); ZHEJIANG UNIVERSITY OF TECHNOLOGY, Hangzhou (CN); ZHEJIANG ZHULONG BIOTECHNOLOGY CO., LTD., Huzhou (CN)

(21) Appl. No.: 19/401,428

(22) Filed: Nov. 26, 2025

Related U.S. Application Data
(63) Continuation of application No. PCT/CN2025/094234, filed on Jul. 17, 2025.

(30) Foreign Application Priority Data
Oct. 29, 2024 (CN) 202411520312.5

(51) Int. Cl. C12P 17/18; C12N 9/10

(52) U.S. Cl. CPC C12P 17/182; C12N 9/1007; C12Y 201/01013

(57) ABSTRACT

A tetrahydrofolate methyltransferase mutant, its encoding gene, and applications thereof are provided. The amino acid sequence of a tetrahydrofolate methyltransferase RcoDmdA mutant is as shown in SEQ ID NO: 3. The RcoDmdA mutant possesses higher catalytic activity, allows the substrate THF feeding amount to be significantly increased to 15-20 g/L, increases the product L-5-MTHF yield to 15-17 g/L, and has high substrate conversion rate. The tetrahydrofolate methyltransferase mutant and its encoding gene have important industrial application value.

### CROSS REFERENCE TO THF RELATED APPLICATIONS

This application is a continuation application of International Application No. PCT/CN2025/094234, filed on Jul. 17, 2025, which is based upon and claims priority to Chinese Patent Application No. 202411520312.5, filed on Oct. 29, 2024, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBHZQK002_Sequence_Listing.xml, created on Nov. 25, 2025, and is 89,165 bytes in size.

### TECHNICAL FIELD

The present invention belongs to the fields of genetic engineering and enzyme engineering, and specifically relates to a tetrahydrofolate methyltransferase mutant, its encoding gene, and applications thereof.

### BACKGROUND

L-5-Methyltetrahydrofolate (L-5-MTHF) is the natural active form of folic acid (FA) and is the main folate component in the circulatory system, which can be directly absorbed and utilized. It has currently been recommended as a substitute for synthetic folic acid. The advantage of L-5-MTHF as a nutritional supplement lies in its higher bioavailability; it can be directly absorbed and utilized without metabolic conversion, thereby bypassing the absorption and utilization deficiencies of synthetic folic acid caused by polymorphisms in the DHFR and MTHFR genes. Active folate L-5-MTHF has the properties of natural folate and also avoids the problem of accumulation of unmetabolized synthetic folic acid in the blood.

Due to the high bioavailability of L-5-MTHF, its stable form, calcium L-5-methyltetrahydrofolate, was recognized by the US FDA in 2001 and approved as a new ingredient for use in nutritional health products. Subsequently, it entered the international market as a safer folic acid food additive. Currently, L-5-MTHF, as an innovative folic acid-based pharmaceutical, is gradually replacing chemically synthesized FA and is widely used in the fields of medicine, food, and animal husbandry. Existing commercial L-5-MTHF products are prepared by chemical methods, starting from synthetic folic acid, which is first hydrogenated to produce tetrahydrofolate, followed by crystallization and resolution to obtain (6S, αS)-tetrahydrofolate, and further methylation and reduction reactions to yield the physiologically active (6S, αS)-5-MTHF (L-5-MTHF). This process is complex, costly, and contains impurities of (6R, αS)-5-MTHF, which lacks physiological activity.

With the rapid development of biotechnology, environmentally friendly, efficient, and low-cost production methods have gradually attracted attention. Among them, the green synthesis technology for catalytic production of L-5-MTHF via biocatalysis has been continuously explored and improved, including microbial fermentation (Microb. Biotechnol. 2022, 15:2758-2772, J. Agric. Food Chem. 2022, 70:5849-5859) and enzymatic catalysis (CN116574768A). Enzymatic methods possess advantages such as high catalytic efficiency, high selectivity, mild reaction conditions, low energy consumption, and non-toxicity, aligning with green development trends. Therefore, biocatalytic processes based on highly active enzymes have significant application value.

A publicly available tetrahydrofolate methyltransferase mutant derived from Ruegeria conchae is now disclosed, capable of efficiently catalyzing the methylation of tetrahydrofolate (THF) to produce L-5-MTHF, exhibiting high substrate feeding levels and fast catalytic reaction rates.

### SUMMARY

To overcome the problems in the prior art such as low catalytic efficiency, low yield, and low conversion rate, the present invention provides a tetrahydrofolate methyltransferase mutant, its encoding gene, and applications thereof.

To achieve the above objectives, the present invention is implemented through the following technical solutions:

First aspect, the present invention provides a tetrahydrofolate methyltransferase RcoDmdA mutant, wherein an amino acid sequence of the mutant is as shown in any one of SEQ ID NOS: 3 to 9.

The present invention performs mutagenesis on a tetrahydrofolate methyltransferase RcoDmdA derived from Ruegeria conchae, aiming to enhance its catalytic activity. The amino acid sequence of RcoDmdA is as shown in SEQ ID NO: 2.

The present invention targets amino acid residue sites within the tetrahydrofolate (THF) binding domain of RcoDmdA, including positions V121, P150, S196, F245, M249, and F263, as well as sites within the dimethylsulfoniopropionate (DMSP) methyl donor binding domain, including positions Y29, C60, and G249, for mutagenesis.

The present invention utilizes computer-aided software to perform homology modeling on the wild-type RcoDmdA and compares it with known homologous crystal structures, specifically the tetrahydrofolate methyltransferase PubDmdA from Pelagibacter ubique (PDB ID: 3TFI and 3TFJ), to identify and screen key amino acid residue sites, as shown in FIG. 2. Site-directed mutagenesis is performed to obtain mutant enzymes, and superior mutants are selected through screening.

The present invention selects 13 amino acid residues within 5 Å of the THF binding pocket of RcoDmdA, including positions Y29, Y93, P107, V121, A122, P150, F175, F176, S193, G194, L242, L243, and F263, and constructs a small but smart combinatorial mutagenesis library using a trinucleotide saturation mutagenesis technique to screen and obtain superior mutants.

The present invention divides the above 13 selected amino acid residues into four groups: Group A (Y29, Y93, P107), Group B (V121, A122, F175, F176), Group C (P150, S193, G194), and Group D (L242, L243, F263), and constructs four small but smart libraries according to the scheme shown in FIG. 4. The three selected codons are isoleucine, serine and tyrosine, and they are used for trinucleotide saturation mutagenesis at each site. Superior mutants are obtained through screening of each mutagenesis library.

The amino acid sequences of the mutants provided by the present invention are as shown in SEQ ID NOS: 3 to 9, corresponding to RcoDmdA-F263Y, RcoDmdA-V121I, RcoDmdA-A122S, RcoDmdA-V121S, RcoDmdA-P107I, RcoDmdA-F263I, and RcoDmdA-F245Y, respectively.

Second aspect, the present invention provides a encoding gene of the tetrahydrofolate methyltransferase RcoDmdA mutant.

As a preferred embodiment, the nucleotide sequence of the encoding gene is as shown in any one of SEQ ID NOS: 10 to 16, encoding RcoDmdA-F263Y, RcoDmdA-V121I, RcoDmdA-A122S, RcoDmdA-V121S, RcoDmdA-P107I, RcoDmdA-F263I, and RcoDmdA-F245Y, respectively.

The encoding gene of the superior mutant F263Y can be synthesized after codon optimization based on its amino acid sequence. Preferably, the nucleotide sequence of the encoding gene is as shown in SEQ ID NO: 10.

Third aspect, the present invention further provides a recombinant vector containing the encoding gene.

Fourth aspect, the present invention further relates to a genetically engineered bacterium containing the encoding gene.

Fifth aspect, the present invention further provides the use of the tetrahydrofolate methyltransferase RcoDmdA mutant in the enzymatic catalysis of tetrahydrofolate (THF) for the preparation of L-5-methyltetrahydrofolate.

As a preferred embodiment, the use involves constructing a genetically engineered microorganism containing the encoding gene of the mutant, using wet cells obtained from fermentation culture of the genetically engineered microorganism or cell lysate obtained by cell disruption as a catalyst to methylate THF and obtain L-5-MTHF.

The present invention can clone the genes of the RcoDmdA mutants F263Y, V121I, A122S, V121S, P107I, F263I, and F245Y into an expression plasmid and transform them into a host cell. After induction and fermentation, the enzyme catalyst is obtained for catalyzing the conversion of THF to Z-5-MTHF.

As a preferred embodiment, the pET28a plasmid and E. coli BL21 host cells are used to construct the recombinant strain E. coli BL21.

The expression plasmid and host cell used in the present invention are preferably the pET28a plasmid and E. coli BL21 (DE3) host cells, i.e., constructing recombinant strains E. coli BL21 (DE3) (pET28a-RcoDmdA-F263Y, pET28a-RcoDmdA-V121I, pET28a-RcoDmdA-A122S, pET28a-RcoDmdA-V121S, pET28a-RcoDmdA-P107I, pET28a-RcoDmdA-F263I, pET28a-RcoDmdA-F245Y).

As a preferred embodiment, the catalytic reaction is carried out in the presence of the methyl donor dimethylsulfonium chloride.

The method of the present invention using wet cells or supernatant obtained from cell disruption to catalyze the conversion of THF to L-5-MTHF by adding a cell density OD600 of 20 to 80 (preferably 40), a substrate THF feeding amount of 1 g/L to 20 g/L (preferably 10-15 g/L), and methyl donor dimethylsulfonium chloride feeding amount of 10 mM to 300 mM (preferably 150 mM).

As a preferred embodiment, the catalytic reaction is conducted at a pH of 6.0-9.0 and a temperature of 25° C.-50° C., whereby the product L-5-MTHF can be obtained.

The catalytic reaction is conducted at the pH of 6.0-9.0 (preferably 7.5), the temperature of 25° C.-50° C. (preferably 37° C.), whereby the product L-5-MTHF can be obtained, and the conversion rate reaches 85%-99%.

The beneficial effects of the present invention are mainly reflected in:
(1) The RcoDmdA mutant provided by the present invention exhibits higher catalytic activity;
(2) The substrate THF feeding amount is significantly increased to 15-20 g/L, the yield of product L-5-MTHF is increased to 15-17 g/L, and the substrate conversion rate is high;
(3) The tetrahydrofolate methyltransferase mutant and its encoding gene provided by the present invention have significant industrial application value.

### BRIEF DESCRIPTION OF THF DRAWINGS

FIG. 1 is a schematic diagram of the structure of the wild-type tetrahydrofolate methyltransferase RcoDmdA.

FIG. 2 is a schematic diagram showing amino acid residue sites around the THF and methyl donor DMSP binding pockets of the wild-type tetrahydrofolate methyltransferase RcoDmdA.

FIG. 3 is a schematic diagram showing 13 unsaturated amino acid sites within 5 Å of THF around the wild-type tetrahydrofolate methyltransferase RcoDmdA.

FIG. 4 is a schematic diagram showing the grouping and construction of small but smart libraries for trinucleotide saturation mutagenesis.

FIG. 5 is a schematic diagram showing the initial screening results of the Group A mutant library in the first-generation trinucleotide saturation mutagenesis library.

FIG. 6 is a schematic diagram showing the initial screening results of the Group B mutant library in the first-generation trinucleotide saturation mutagenesis library.

FIG. 7 is a schematic diagram showing the initial screening results of the Group C mutant library in the first-generation trinucleotide saturation mutagenesis library.

FIG. 8 is a schematic diagram showing the initial screening results of the Group D mutant library in the first-generation trinucleotide saturation mutagenesis library.

FIG. 9 is a comparative plot of product synthesis curves for single-point mutants in the catalytic preparation of L-5-MTHF from THF.

FIG. 10 is an HPLC chromatogram of THF and L-5-MTHF standard samples.

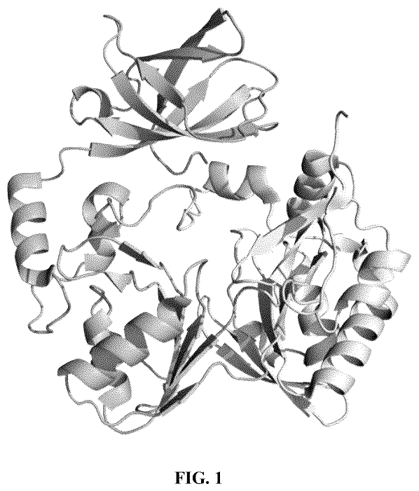

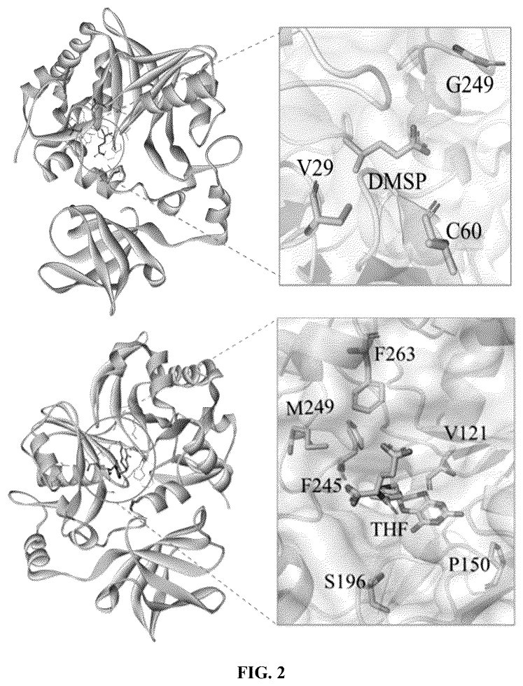

### DETAILED DESCRIPTION OF THF EMBODIMENTS

The following specific embodiments are used to illustrate the implementation modes of the present invention. Skilled persons in the art can easily understand other advantages and effects of the present invention from the content disclosed in this specification. The present invention may also be implemented or applied in different specific ways, and various modifications or changes may be made to the details of this specification based on different viewpoints and applications without departing from the spirit of the present invention. It should be noted that, unless otherwise specified, the methods used in the examples are conventional methods, and the reagents used can be commercially obtained.

The following will further describe the present invention in detail with reference to specific examples, which are only used to explain the present invention and do not limit the protection scope of the present invention.

LB medium: yeast extract 5.0 g/L, peptone 10.0 g/L, NaCl 10.0 g/L, solvent is distilled water.

Fermentation medium: yeast extract 12.0 g/L, peptone 15.0 g/L, NazHPO₄·12H₂O 8.9 g/L, KH₂PO₄ 3.4 g/L, NH₄Cl 2.67 g/L, Na₂SO₄ 0.71 g/L, MgSO₄ 7H₂O 0.49 g/L, kanamycin 50 μg/L, pH 7.0, solvent is distilled water.

400 mmol/L HEPES buffer solution (pH 7.5): HEPES 104.1 g, solvent is distilled water.

### Example 1 Construction of Recombinant Tetrahydrofolate Methyltransferase RcoDmdA Expression Strain and its Induced Expression

The tetrahydrofolate methyltransferase RcoDmdA derived from Ruegeria conchae was retrieved from the GenBank database, i.e., GenBank accession No. UWR04622, with its amino acid sequence as shown in SEQ ID NO: 2. After codon optimization, the RcoDmdA encoding gene sequence was obtained, as shown in SEQ ID NO: 1. The gene was submitted to Genewiz for gene synthesis and cloned into the pET28a plasmid to obtain the recombinant expression plasmid pET28a-RcoDmdA. The plasmid was transformed into the expression host Escherichia coli BL21 (DE3) strain to obtain the recombinant strain E. coli BL21 (DE3) (pET28a-RcoDmdA).

The recombinant strain was streaked from the preserved glycerol stock onto an LB plate and cultured overnight. A single colony was inoculated into LB liquid medium containing 50 μg/mL kanamycin and cultured overnight at 37° C., 200 rpm. It was then inoculated into the fermentation medium at 1-2% (v/v) inoculation amount, cultured at 37° C. for 2 h; IPTG was added to a final concentration of 0.5 mmol/L, the cultivation temperature was adjusted to 24° C., and fermentation was continued for 10 h to obtain a bacterial agent overexpressing RcoDmdA.

### Example 2 Design of RcoDmdA Single-Point Mutations and Construction of Mutant Sites

To improve the catalytic reaction rate and product yield of RcoDmdA, mutation sites were screened using a homologous sequence alignment method. The three-dimensional structure was compared and analyzed with known homologous enzymes, specifically the crystal structures of PubDmdA from Pelagibacter ubique, including PDB IDs 3TFH, 3TFI, and 3TFJ. Through comparative analysis of the structural domains of the two enzymes, nine differing amino acid residues were identified around the THF and DMSP binding pockets, including positions 29, 60, 121, 150, 196, 245, 246, 249, and 263, and site-directed mutagenesis was performed at these sites. Based on the wild-type RcoDmdA gene sequence shown in SEQ ID NO: 1, primers for site-directed mutagenesis were designed, as shown in Table 1.

Each site on RcoDmdA was mutated to the corresponding amino acid residue of PubDmdA. The site-directed mutagenesis was constructed as follows: using the plasmid pET28a-RcoDmdA as a template, PCR amplification was performed with primers introducing the desired mutation site. The amplified product was purified using a PCR purification kit, digested with DpnI, and the digested product was subjected to a ligation reaction using the one-step cloning kit ClonExpress™ II from Vazyme. The ligation product was transformed into E. coli BL21 (DE3) cells, plated on LB plates containing 50 μg/mL kanamycin, and verified by colony PCR and sequencing, thereby obtaining site-directed mutants for the 9 sites.

TABLE 1
Primers for Site-Directed Replacement Mutation

| | | SEQ ID |
|---|---|---|
| Mutants | Primers (5′-3′) | NO: |
| V29I-F | GGAAGGCGTGAAAGGCTATACCATTTATAACCACA | 17 |
| V29I-R | ATAGCCTTTCACGCCTTCCGCTTCCAC | 18 |
| C60A-F | ATGTGCAAGTGTGGGATGTGAGCGCGGAACGCCAAGTG | 19 |
| C60A-R | ATCCCACACTTGCACATGTTTTTTCAGA | 20 |
| S247N-F | AGCGGCCTGCTGAGCTTTGGCAACGATATGCGC | 21 |
| S247N-R | GCTCAGCAGGCCGCTTTCAATGCGT | 22 |
| V121I-F | CTGGCGGATGATCATTATTGGCTGAGCATTGCGGATGGCGATC | 23 |
| V121I-R | AATAATGATCATCCGCCAGTTTAATCGCCACCGGATC | 24 |
| P150I-F | GTGAGCGAACCGGATGTGAGCATTCTGGCGGTGCAAGGC | 25 |
| P150I-R | ACATCCGGTTCGCTCACTTCCACATCCAGTT | 26 |
| S196Q-F | TGTGATTGCGCGCAGCGGCTGGCAAAAACAAGGCGGC | 27 |
| S196R-R | CGCTGCGCGCAATCACAAAGCTCGTATCT | 28 |
| F245Y-F | GCATTGAAAGCGGCCTGCTGAGCTATGGCAGCGAT | 29 |
| F245Y-R | CAGCAGGCCGCTTTCAATGCGTTCAATGCCGTTCGGGCA | 30 |
| M249P-F | AGCGGCCTGCTGAGCTTTGGCAGCGATTTTCGCCGCGAAA | 31 |
| M249P-R | TGCCAAAGCTCAGCAGGCCGCTTTCAATGCGTTCAATG | 32 |
| F263Y-F | CCCCGTATGAATGCGGCCTGGAACGCTATTGCAACAGCC | 33 |
| F263Y-R | CCAGGCCGCATTCATACGGGGTGTTTTCGCGGCGC | 34 |

### Example 3 Screening and Activity Comparison of Single-Point Mutants

The transformants from the plates obtained in Example 2 were transferred to LB liquid medium containing 50 μg/mL kanamycin and cultured in a shaker until mid-logarithmic phase. They were then inoculated into fermentation medium at 1-2% (v/v), cultured at 37° C. for 2 h, induced with 0.5 mmol/L IPTG, and fermentation was continued at 24° C. for 10 h to obtain the induced bacterial agent.

Following the method described in Example 1, bacterial agents containing the RcoDmdA mutants were prepared, 50 mL for each agent. Cells were collected by centrifugation at 10000×g for 10 min, and the cells were resuspended in 10 mL of 0.4 mol/L HEPES buffer (pH 7.5) for ultrasonic disruption, and the supernatant was collected by centrifugation. THF was added to a final concentration of 10.0 g/L, dimethylsulfonium chloride (MSDS) to 100.0 mmol/L, and DTT to 1.0 g/L. The catalytic reaction was carried out in a 37° C. magnetic stirrer water bath for 10 h, and the catalytic reaction solution was used for HPLC analysis.

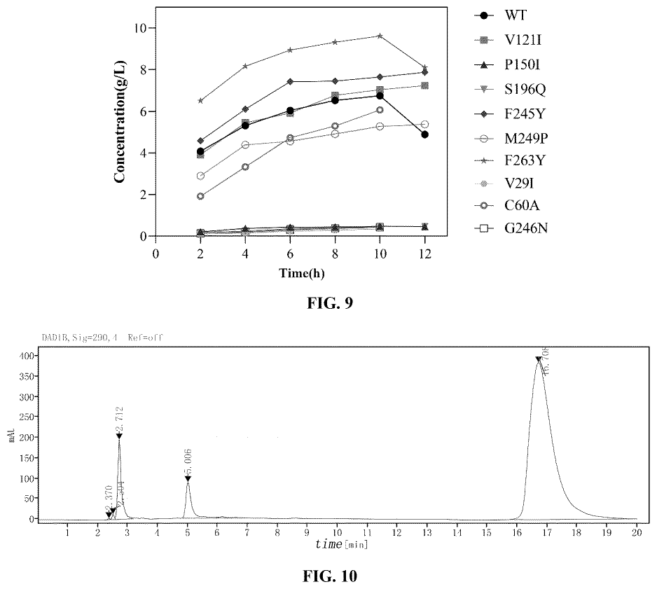

The initial screening results of the single-point mutants at various time points are shown in FIG. 9.

The catalytic results of the nine single-point mutants are shown in Table 2. By comparing the substrate conversion rates, the preferred mutants F245Y, V121I, and F263Y were obtained.

TABLE 2
Screening Results of Single-Point Mutants

| Mutants | Conv. (%) |
|---|---|
| WT | 61.0 |
| F263Y | 96.1 |
| F245Y | 78.7 |
| V121I | 72.3 |
| C60A | 60.6 |
| V29I | 3.4 |
| S247N | 4.3 |
| P150I | 4.7 |
| S196Q | 4.6 |
| M249P | 53.7 |

### Example 4 Design of RcoDmdA Mutation Sites and Construction of Small but Smart Mutagenesis Libraries

To further optimize the catalytic pocket of RcoDmdA and improve its catalytic reaction rate and product yield, trinucleotide saturation mutagenesis was performed on the THF binding domain of RcoDmdA. A computer-aided design method was used to screen mutation sites. Based on the reported crystal structure of PubDmdA from Pelagibacter ubique (PDB ID: 3TFH), homology modeling was performed on RcoDmdA, and molecular docking software was used. Considering the docking results of RcoDmdA with substrate THF, the characteristics of the enzyme's substrate binding pocket, and the catalytic mechanism, 13 amino acid residue sites were ultimately determined: positions 29, 93, 107, 121, 122, 150, 175, 176, 193, 194, 242, 243, and 263 in the amino acid sequence of SEQ ID NO: 2. The specific positions are shown in FIG. 3, and the grouping is shown in FIG. 4 (different colors indicate different groups: green: Group A; orange: Group B; blue: Group C; purple: Group D). They were divided into Group A (Y29, Y93, P107), Group B (V121, A122, F175, F176), Group C (P150, S193, G194), and Group D (L242, L243, F263).

Based on the wild-type RcoDmdA gene sequence shown in SEQ ID NO: 1, primers for trinucleotide saturation mutagenesis of Groups A, B, C, and D were designed, as shown in Table 3. The three codons correspond to serine, isoleucine, and tyrosine. The construction method for the trinucleotide saturation mutagenesis library is as follows: first, PCR amplification of the fragment at the mutation site was performed, with random mutations introduced via primers, followed by gel purification; then, the purified PCR product was used as one primer, and the recombinant plasmid pET28a-RcoDmdA as template, to further amplify the entire plasmid. After 1% agarose gel electrophoresis, the correctly sized band was obtained. The PCR product was digested with DpnI, subjected to a one-step cloning ligation reaction, and then transformed into E. coli BL21 (DE3) cells, plated on LB plates containing 50 μg/mL kanamycin, to obtain the trinucleotide saturation mutagenesis library.

TABLE 3
Primers for Trinucleotide Saturation Mutagenesis

| | | SEQ ID |
|---|---|---|
| Mutants | Primers (5′-3′) | NO: |
| 121122WT-F | TGATCATTATTGGCTGAGCGTGGCGGATGGCGATCTG | 35 |
| 121AKT-F | TGATCATTATTGGCTGAGCAKTGCGGATGGCGATCTG | 36 |
| 121TAT-F | TGATCATTATTGGCTGAGCTATGCGGATGGCGATCTG | 37 |
| 122AKT-F | TGATCATTATTGGCTGAGCGTGAKTGATGGCGATCTG | 38 |
| 122TAT-F | TGATCATTATTGGCTGAGCGTGTATGATGGCGATCTG | 39 |
| 121122AKT-F | TGATCATTATTGGCTGAGCAKTAKTGATGGCGATCTG | 40 |
| 121122TAT-F | TGATCATTATTGGCTGAGCTATTATGATGGCGATCTG | 41 |
| 121AKT122TAT-F | TGATCATTATTGGCTGAGCAKTTATGATGGCGATCTG | 42 |
| 121TAT122AKT-F | TGATCATTATTGGCTGAGCTATAKTGATGGCGATCTG | 43 |
| 175176WT-R | CGTTTATAGCGAAAAAATTTAATATCGCGCA | 44 |
| 175TMA-R | CGTTTATAGCGAAATMATTTAATATCGCGCA | 45 |
| 175ATA-R | CGTTTATAGCGAAAATATTTAATATCGCGCA | 46 |
| 176TMA-R | CGTTTATAGCGTMAAAATTTAATATCGCGCA | 47 |
| 175176TMA-R | CGTTTATAGCGTMATMATTTAATATCGCGCA | 48 |
| 175176ATA-R | CGTTTATAGCGATAATATTTAATATCGCGCA | 49 |
| 175TMA176ATA-R | CGTTTATAGCGTMAATATTTAATATCGCGCA | 50 |
| 175ATA176TMA-R | CGTTTATAGCGATATMATTTAATATCGCGCA | 51 |
| CZ121-R | CAGCCAATAATGATCATCCGCCAGTTTAAT | 52 |
| 150/WT-F | GTGAGCGAACCGGATGTGAGCCCGCTGGCGGTGCAA | 53 |
| | G | |
| 150AKT-F | GTGAGCGAACCGGATGTGAGCAKTCTGGCGGTGCAA | 54 |
| | G | |
| 150TAT-F | GTGAGCGAACCGGATGTGAGCTATCTGGCGGTGCAA | 55 |
| | G | |
| 193194WT-R | CTTGTTTGCTCCAGCCGCTGCGCGCAATCACA | 56 |
| 193ATA-R | CTTGTTTGCTCCAGCCATAGCGCGCAATCACA | 57 |
| 193TAA-R | CTTGTTTGCTCCAGCCAATGCGCGCAATCACA | 58 |
| 194TMA-R | CTTGTTTGCTCCAAMTGCTGCGCGCAATCACA | 59 |
| 194ATA-R | CTTGTTTGCTCCAATAGCTGCGCGCAATCACA | 60 |
| 193ATA194TMA-R | CTTGTTTGCTCCAAMTATAGCGCGCAATCACA | 61 |
| 193TAA194ATA-R | CTTGTTTGCTCCAATAAATGCGCGCAATCACA | 62 |
| 193194ATA-R | CTTGTTTGCTCCAATAATAGCGCGCAATCACA | 63 |
| 193194TMA-R | CTTGTTTGCTCCAAMTAATGCGCGCAATCACA | 64 |
| CZ150-R | TCCGGTTCGCTCACTTCCACATCCAGTTCCA | 65 |
| 242243WT-F | CATTGAACGCATTGAAAGCGGCCTGCTGAGCTTTGGC | 66 |
| | AGC | |
| 242AKT-F | CATTGAACGCATTGAAAGCGGCAKTCTGAGCTTTGGC | 67 |
| | AGC | |
| 242TAT-F | CATTGAACGCATTGAAAGCGGCTATCTGAGCTTTGGC | 68 |
| | AGC | |
| 243 AKT-F | CATTGAACGCATTGAAAGCGGCCTGAKTAGCTTTGGC | 69 |
| | AGC | |
| 243TAT-F | CATTGAACGCATTGAAAGCGGCCTGTATAGCTTTGGC | 70 |
| | AGC | |
| 242AKT243TAT-F | CATTGAACGCATTGAAAGCGGCAKTTATAGCTTTGGC | 71 |
| | AGC | |
| 242TAT243AKT-F | CATTGAACGCATTGAAAGCGGCTATAKTAGCTTTGGC | 72 |
| | AGC | |
| 242243TAT-F | CATTGAACGCATTGAAAGCGGCTATTATAGCTTTGGC | 73 |
| | AGC | |
| 242243 AKT-F | CATTGAACGCATTGAAAGCGGCAKTAKTAGCTTTGGC | 74 |
| | AGC | |
| 263WT-R | CGGGCTGTTGCAAAAGCGTTCCAGGCCGC | 75 |
| 263TMA-R | CGGGCTGTTGCAAMTGCGTTCCAGGCCGC | 76 |
| 263 ATA-R | CGGGCTGTTGCAATAGCGTTCCAGGCCGC | 77 |
| CZ242-R | GCTTTCAATGCGTTCAATGCCGTTCGGGCA | 78 |
| 29AKT-F | GGAAGGCGTGAAAGGCTATACCGTGAKTAACCACAT | 79 |
| | GC | |
| 93107WT-R | GTTTAATCGCCACCGGATCGTTCAGCATGCCGCCGTT | 80 |
| | CTGATCCACAATCGGCACATAATAGCACTGATC | |
| 93107TMA-R | GTTTAATCGCCACAMTATCGTTCAGCATGCCGCCGTT | 81 |
| | CTGATCCACAATCGGCACAMTATAGCACTGATC | |
| 107ATA-R | GTTTAATCGCCACATAATCGTTCAGCATGCCGCCGTT | 82 |
| | CTGATCCACAATCGGCACATAATAGCACTGATC | |
| 93TMA107ATA-R | GTTTAATCGCCACATAATCGTTCAGCATGCCGCCGTT | 83 |
| | CTGATCCACAATCGGCACAMTATAGCACTGATC | |
| 93TMA-R | GTTTAATCGCCACCGGATCGTTCAGCATGCCGCCGTT | 84 |
| | CTGATCCACAATCGGCACAMTATAGCACTGATC | |
| 107TMA-R | GTTTAATCGCCACAMTATCGTTCAGCATGCCGCCGTT | 85 |
| | CTGATCCACAATCGGCACATAATAGCACTGATC | |
| CZ29-R | AGCCTTTCACGCCTTCCGCTTCCACGCCCG | 86 |

### Example 5 Screening of the Four Small but Smart Mutant Libraries

The trinucleotide saturation mutagenesis libraries obtained in Example 4 were used. Antimicrobial transformants growing on the plates were transferred to LB liquid medium containing 50 μg/mL kanamycin, cultured until mid-logarithmic phase. They were then inoculated into fermentation medium at 1-2% (v/v), cultured at 37° C. for 2 h, induced with 0.5 mmol/L IPTG, and fermentation was continued at 24° C. for 10 h to obtain the induced bacterial agent.

2 mL of the cultured transformants were centrifuged at 10,000×g for 5 min to collect cells. The cells were resuspended in 1 mL of 0.4 mol/L HEPES buffer (pH 7.5), and 5.0 g/L THF and 50 mmol/L MSDS were added. The reaction was carried out at 37° C., 1000 rpm in a metal bath for 2 h, and the reaction mixture was analyzed by HPLC.

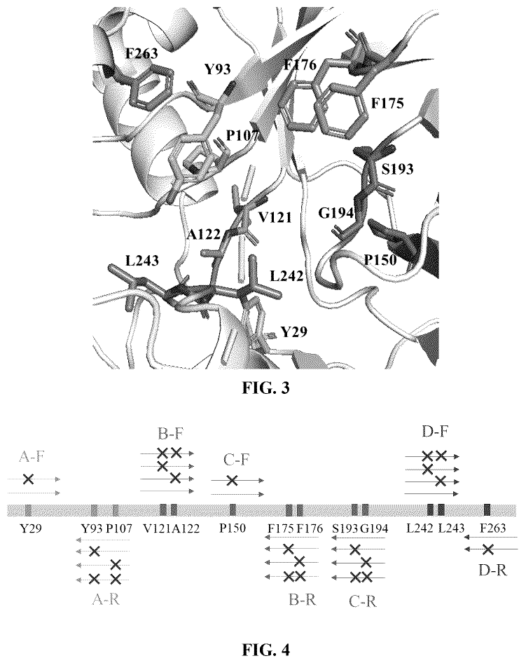

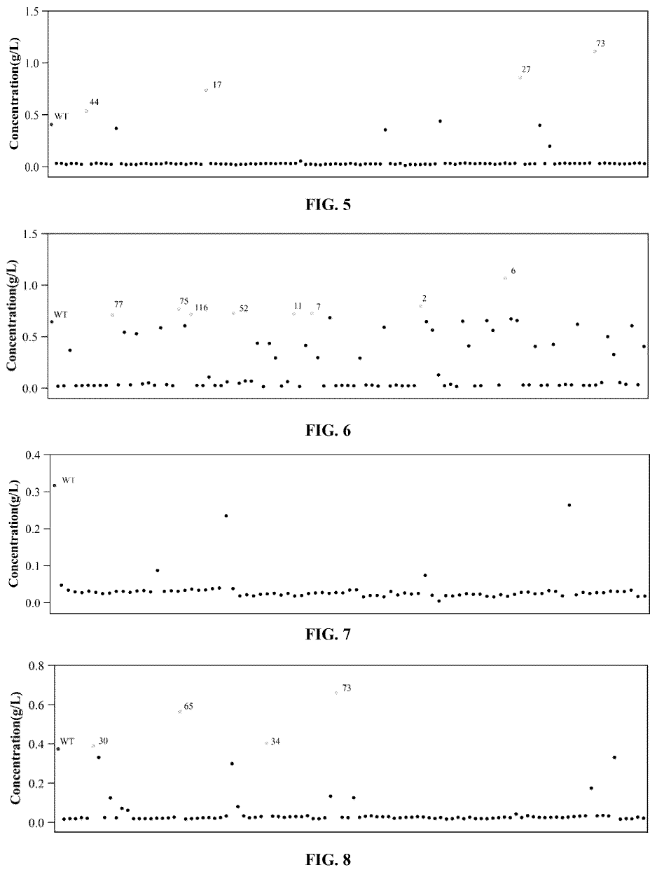

The enzyme activity screening results of the mutants in libraries LibA, LibB, LibC, and LibD are shown in FIGS. 5, 6, 7, and 8, respectively.

By comparing the substrate conversion rates, superior mutants were selected for sequencing analysis, and the mutants F263Y, V121I, P107I, V121S, A122S, and F263I were obtained as the preferred mutants from the first-generation library screening.

### Example 6 Activity Analysis of First-Generation Preferred Mutants in Catalyzing THF to Prepare L-5-MTHF

The bacterial agents of RcoDmdA mutants F263Y, V121I, V121S, A122S, F263I, and P107I were prepared as described in Example 1. Each 30 mL bacterial agent was centrifuged at 10,000×g for 10 min to collect cells. The cells were resuspended in 10 mL of 0.4 mol/L HEPES buffer (pH 7.5). 20.0 g/L THF, 100.0 mmol/L MSDS, and 1.0 g/L DTT were added. The reaction was carried out at 37° C. in a magnetic stirrer water bath for 10 h, and the reaction mixture was analyzed by HPLC.

The re-screening results of the first-generation preferred mutants are shown in Table 4. Among them, the mutant F263Y exhibited the best catalytic activity toward substrate THF and was identified as the first-generation optimal mutant.

TABLE 4
Re-screening Results of First-Generation Preferred Mutants

| Mutants | Conv. (%) |
|---|---|
| WT | 29.4 |
| F263Y | 72.5 |
| F263I | 63.0 |
| V121I | 38.0 |
| V121S | 29.6 |
| A122S | 33.5 |
| P107I | 46.4 |

### Example 7 Application of the Superior Mutant RcoDmdA-F263Y in Catalyzing THF to Prepare Z-5-MTHF

Induced expression of RcoDmdA mutants. The recombinant strain E. coli BL21 (DE3) (pET28a-RcoDmdA-F263Y) was streaked onto an LB plate from the preserved glycerol stock and cultured overnight. A single colony was inoculated into LB liquid medium containing 50 μg/mL kanamycin and cultured overnight at 37° C., 200 rpm. The seed culture was inoculated at 2% into LB medium containing kanamycin sulfate (50 μg/mL), cultured at 37° C., 200 rpm for 4-6 h, and then inoculated at 2% into a 3 L fermenter. Fermentation was carried out at 37° C. When the OD600 reached about 10, 10 g/L lactose was added to induce the expression of the target protein. The induction temperature was 22° C., and induction was continued for 8 h before ending fermentation, yielding the bacterial agent for catalytic reaction.

Catalytic reaction with 1 g/L THF feeding. Cells were collected by centrifugation, resuspended in 0.4 mol/L HEPES buffer (pH 7.5) to an OD600 of 40, and subjected to high-pressure homogenization to obtain cell lysate. 30 mL was added to a round-bottom flask, pre-incubated at 37° C. in a water bath for 10 min, and then 1 g/L THF, 20 mmol/L MSDS, and 1.0 g/L DTT were added sequentially. Magnetic stirring was started immediately to mix uniformly, and the reaction start time was recorded. After 1 h, the L-5-MTHF yield was 1.052 g/L, with a conversion rate of 102%.

Catalytic reaction with 10 g/L THF feeding. Cells were collected by centrifugation, resuspended in 0.4 mol/L HEPES buffer (pH 7.5) to an OD600 of 40, and subjected to high-pressure homogenization to obtain cell lysate. 30 mL was added to a round-bottom flask, pre-incubated at 37° C. in a water bath for 10 min, and then 10 g/L THF, 100 mmol/L MSDS, and 1.0 g/L DTT were added sequentially. Magnetic stirring was started immediately to mix uniformly, and the reaction start time was recorded. After 5 h, the L-5-MTHF yield was 9.9 g/L, with a conversion rate of 99%.

Catalytic reaction with 15 g/L THF feeding. Cells were collected by centrifugation, resuspended in 0.4 mol/L HEPES buffer (pH 7.5) to an OD600 of 40, and subjected to high-pressure homogenization to obtain cell lysate. 30 mL was added to a round-bottom flask, pre-incubated at 37° C. in a water bath for 10 min, and then 15 g/L THF, 150 mmol/L MSDS, and 1.0 g/L DTT were added sequentially. Magnetic stirring was started immediately to mix uniformly, and the reaction start time was recorded. After 10 h, the L-5-MTHF yield was 14.9 g/L, with a conversion rate of 99.4%.

Catalytic reaction with 20 g/L THF feeding. Cells were collected by centrifugation, resuspended in 0.4 mol/L HEPES buffer (pH 7.5) to an OD600 of 40, and subjected to high-pressure homogenization to obtain cell lysate. 30 mL was added to a round-bottom flask, pre-incubated at 37° C. in a water bath for 10 min, and then 20 g/L THF, 150 mmol/L MSDS, and 1.0 g/L DTT were added sequentially. Magnetic stirring was started immediately to mix uniformly, and the reaction start time was recorded. After 12 h, the L-5-MTHF yield was 17.1 g/L, with a conversion rate of 85%.

Catalytic reaction with 30 g/L THF feeding. Cells were collected by centrifugation, resuspended in 0.4 mol/L HEPES buffer (pH 7.5) to an OD600 of 40, and subjected to high-pressure homogenization to obtain cell lysate. 30 mL was added to a round-bottom flask, pre-incubated at 37° C. in a water bath for 10 min, and then 30 g/L THF, 300 mmol/L MSDS, and 1.0 g/L DTT were added sequentially. Magnetic stirring was started immediately to mix uniformly, and the reaction start time was recorded. The conversion rates at 8 h and 13 h were 55.2% and 63.3%, respectively.

What is claimed is:

1. A tetrahydrofolate methyltransferase RcoDmdA mutant, wherein the amino acid sequence of the tetrahydrofolate methyltransferase RcoDmdA mutant is as shown in SEQ ID NO: 3.

2. A gene encoding the tetrahydrofolate methyltransferase RcoDmdA mutant according to claim 1.

3. The gene according to claim 2, wherein the nucleotide sequence of the gene is as shown in SEQ ID NO: 10.

4. A method for preparing L-5-methyltetrahydrofolate (L-5-MTHF) by enzymatic catalysis of tetrahydrofolate (THF), comprising using the tetrahydrofolate methyltransferase RcoDmdA mutant according to claim 1.

5. The method according to claim 4, wherein the method comprises: constructing a genetically engineered bacterium containing a gene of the tetrahydrofolate methyltransferase RcoDmdA mutant, using wet cells obtained by fermentation culture of the genetically engineered bacterium or enzyme-containing cells obtained by cell disruption as a catalyst, to methylate THF, thereby obtaining L-5-MTHF.

6. The method according to claim 5, wherein a pET28a plasmid and E. coli BL21 host cells are used to construct a recombinant bacterium E. coli BL21.

7. The method according to claim 5, wherein a catalytic action is carried out in a presence of methyl donor dimethylsulfonium chloride.

8. The method according to claim 5, wherein a catalytic reaction is carried out at a pH of 6.0-9.0 and a temperature of 25° C.-50° C.

9. The method according to claim 6, wherein a catalytic action is carried out in a presence of methyl donor dimethylsulfonium chloride.

10. The method according to claim 6, wherein a catalytic reaction is carried out at a pH of 6.0-9.0 and a temperature of 25° C.-50° C.
